(19) United States
(12) Patent Application Publication
(10) Pub. No.: US 2009/0105625 A1
(43) Pub. Date: Apr. 23, 2009

(54) Finger and toe protection

(76) Inventors: Frank Kohner, New York, NY (US); Albert Stubbmann, Macungie, PA (US)

Correspondence Address: Kirschstein, Israel, Schiffmiller & Pieroni, P.C., 425 FIFTH AVENUE, 5TH FLOOR, NEW YORK, NY, 10016-2223, US

(21) Appl. No.: 12/012,584

(22) Filed: Feb. 4, 2008

Related U.S. Application Data
(60) Provisional application No. 61/000,083, filed on Oct. 23, 2007.

(51) Int. Cl. A61F 13/00

(52) U.S. Cl. 602/54; 602/59; 602/58

(57) ABSTRACT

A toe and finger guard includes a protective member bendable from an initial, generally planar shape to a curved shape when mounted on a digit to be protected. A foam member is secured to the protective member. An adhesive tape overlies the protective member and the foam member and secures them on the digit. Advantageously, the protective member is a plastic square that is initially rigid and generally planar. The square is deeply grooved to acquire the capacity to be bent from its initial planar, generally rigid shape to a curved, flexible shape when mounted on the digit to be protected.

### CROSS REFERENCE TO RELATED APPLICATION

This application claims the priority of U.S. Provisional Application Ser. No. 61/000,083, filed Oct. 23, 2007.

### BACKGROUND OF THE INVENTION

This invention relates to a guard for protecting injured or wounded digits, such as fingers and/or toes.

It has been recognized that protection of an injured or wounded finger or toe is desirable to permit use of a person's hand or foot without danger of reinjuring the digit or aggravating the wound. A useful device would be one that physically protects the digit from being struck. A further desirable feature would be a barrier to prevent dirt and other impurities from reaching the wound.

### SUMMARY OF THE INVENTION

The guard of this invention distinguishes itself from all other guards for protecting injured digits, e.g., fingers and/or toes, by the provision of a protective member, e.g., a plastic square, that is initially rigid and then deeply grooved to acquire the capacity to be bent from an initial planar, generally rigid shape to a curved, flexible shape when mounted on a digit to be protected. Grooves on the protective member extend almost entirely therethrough and leave a thin outer skin thereon. A foam member is adhesively mounted on the outer skin of the protective member. An adhesive tape overlies the protective member and the foam member and secures them on the digit to be protected. Advantageously, to prevent dirt and other impurities from reaching a wound, a user will be instructed to first attach a standard Band-Aid™ or bandage over the wound, and then subsequently attach the guard over it. Ventilation holes are provided in the protective member.

It is, therefore, an object of the present invention to provide a guard that protects an injured finger or toe from being struck or exposed to impure and undesirable substances.

A further object of the invention is to provide such a guard as may be easily placed on and removed from an injured finger or toe, without pain or discomfort and without agitating the wound.

A still further object of the invention is to provide such a guard that is economical to make and is easy to use.

Yet a further object of the invention is to provide such a guard that will fit comfortably over digits of all sizes, and will readily adjust to the particular contours of such digits.

The above and other features of the invention, including various novel details of construction and combinations of parts, will now be more particularly described with reference to the accompanying drawings. It will be understood that the particular device embodying the invention is shown by way of illustration only and not as a limitation of the invention. The principles and features of this invention may be employed in various and numerous embodiments without departing from the scope of the invention.

### BRIEF DESCRIPTION OF THE DRAWINGS

FIG. 1 is a top plan view of a guard for protecting injured fingers and/or toes in accordance with this invention;

FIG. 2 is a bottom plan view of the guard of FIG. 1;

FIG. 3 is a top plan view of a protective member in isolation;

FIG. 4 is an enlarged sectional view taken in the direction of arrows 4-4 of FIG. 1;

FIG. 5 is a perspective view of the guard being applied to an injured finger that has a wound previously wrapped by a Band-Aid™; and

FIG. 6 is an enlarged sectional view of the guard applied over the Band-Aid™ on the injured finger of FIG. 5.

### DETAILED DESCRIPTION OF THE PREFERRED EMBODIMENT

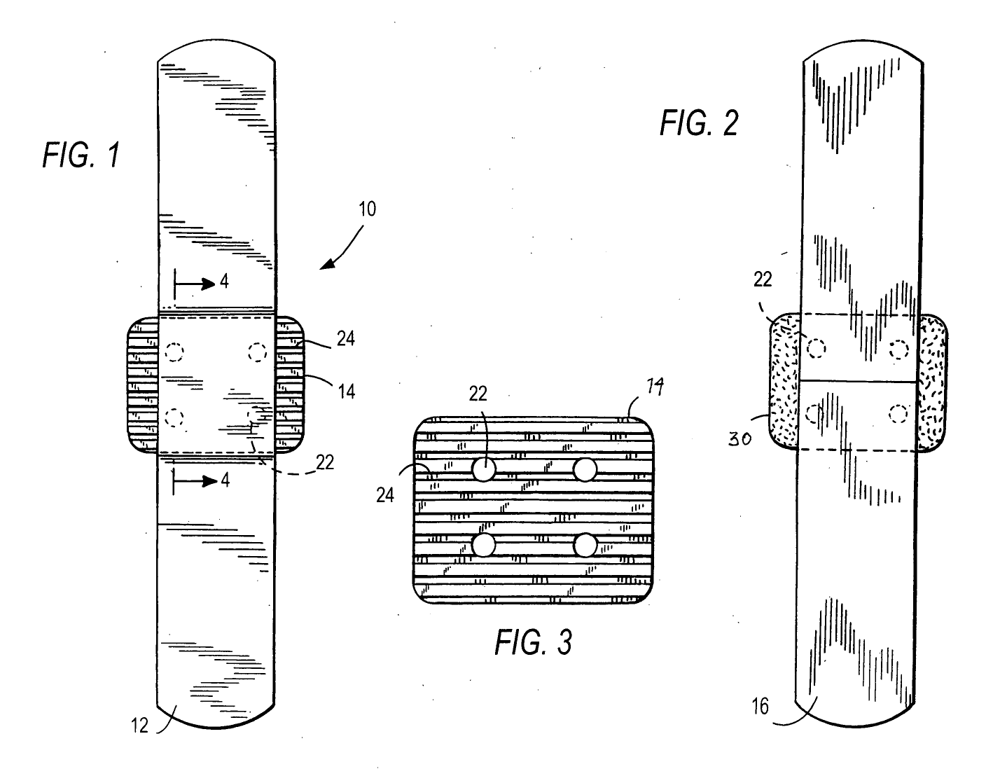

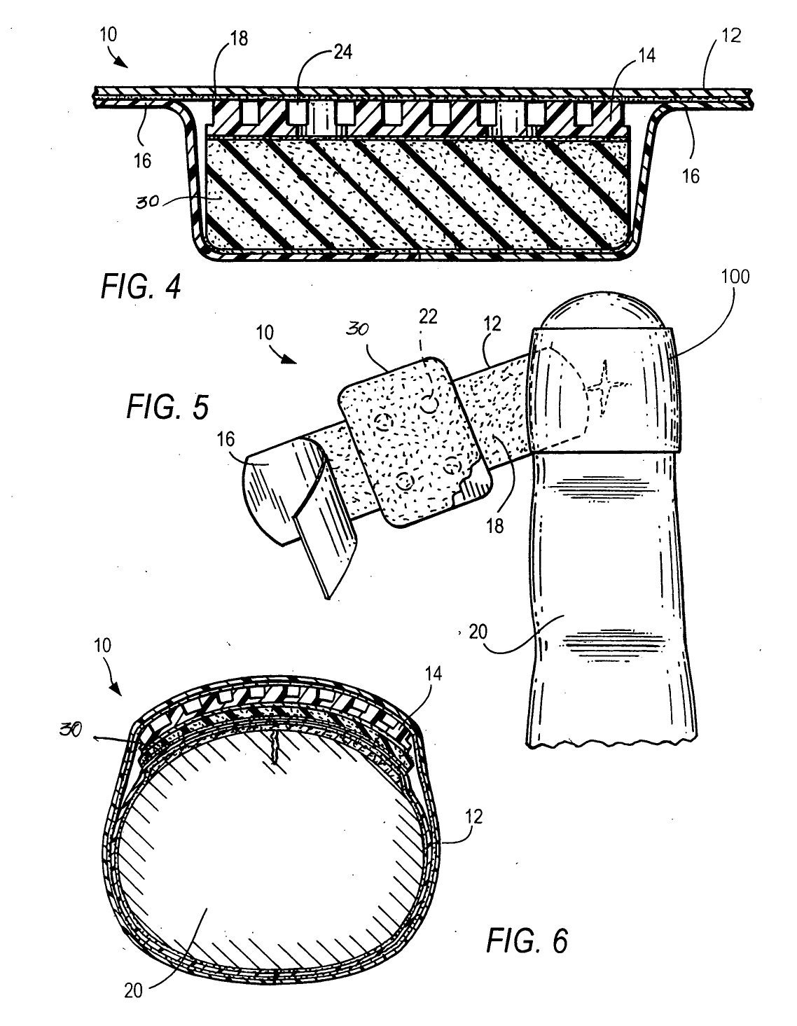

FIG. 1 depicts a top plan view, and FIG. 2 depicts a bottom plan view, of a guard 10 according to this invention. The guard 10 comprises an adhesive tape 12 overlying a protective member 14 (shown in isolation in FIG. 3). As shown in FIG. 4, the protective member 14 is adhered by an adhesive layer 18 to a central area of the tape 12. A foam member 30 is adhered to the protective member 14. A pair of release paper layers 16 is peelably attached to the adhesive layer 18 on the tape 12.

The protective member 14 is a plastic square, preferably with rounded corners. The square 14 is initially rigid and generally planar. The square 14 is deeply grooved to acquire the capacity to be bent from its initial, generally planar, rigid shape to a curved, flexible shape, as depicted in FIG. 6, for mounting on a digit 20 to be protected. Grooves 24 on the square 14 extend almost entirely therethrough and leave a thin outer skin thereon. The foam member 30 is adhesively mounted on the outer skin of the square 14. Any number of grooves 24 will do. The more the number of grooves 24, the greater the flexibility. Advantageously, each groove has a generally rectangular shape. The portions of the square 14, also known as ribs, bounding the grooves are also advantageously each of a rectangular shape. The square 14 is bendable from its initial generally planar shape (FIG. 4) to the curved shape when mounted on the injured digit 20 to be protected.

Initially, the injured digit 20 is preferably wrapped and protected by a Band-Aid™ 100. Then, the paper layers 16 of the guard 10 are peeled away, and the guard 10 with the attached foam member 30 are placed against the Band-Aid™ 100 and secured around the injured digit 20, as shown in FIG. 6. Advantageously, ventilation holes 22 are provided in the protective member 14.

One size of the guard will fit comfortably over fingers and toes of all sizes. The flexibility of the square 14 and the resilience of the foam member 30 provide a perfect fit over any conceivable contour of the fingers and toes, regardless of whether the digit is small or large, or whether the square 14 and the foam member 30 are attached on the top or the side of any digit. The guard is mounted on the injured digit 20 without any pressure and will therefore avoid any pain or discomfort thereto. The foam member 30 helps to absorb and cushion any shock forces from aggravating the wound. If the square 14 and the foam member 30 are too large to fit over a small digit, then they can be cut and trimmed to their required width by a pair of scissors. The guard can be attached with a loose fit, thereby avoiding pressure or pain to the injured digit.

It will be understood that each of the elements described above, or two or more together, also may find a useful application in other types of constructions differing from the types described above.

While the invention has been illustrated and described as a finger and toe protector, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention and, therefore, such adaptations should and are intended to be comprehended within the meaning and range of equivalence of the following claims.

What is claimed as new and desired to be protected by Letters Patent is set forth in the appended claims.

We claim:

1. A guard for protecting a digit, comprising:
a protective member having an initial, generally planar, rigid shape and a plurality of grooves for enabling the protective member to be flexed and bent to a curved shape;
a foam member adhesively mounted on the protective member; and
an adhesive tape on which the protective member and the foam member are mounted, for mounting the foam member and the protective member in the curved shape on, and about, the digit to be protected.

2. The guard of claim 1, wherein the protective member is constituted of a plastic material and has opposite peripheral edges between which the grooves extend in mutual parallelism.

3. The guard of claim 1, wherein each groove has a generally rectangular shape.

4. The guard of claim 1, wherein the protective member has a generally square shape with rounded corners.

5. The guard of claim 1, wherein the protective member has opposite surfaces, and wherein the grooves extend from one of the surfaces through the protective member, and terminate short of the other of the surfaces to form an outer skin.

6. The guard of claim 1, wherein the protective member has ventilation holes extending therethrough.

7. The guard of claim 1, wherein the protective member has generally rectangular ribs bounding each groove.

8. The guard of claim 1, and a pair of peelable strips removably mounted on the adhesive tape.

9. The guard of claim 1, and a bandage wrapped around the digit underneath the protective member, the foam member and the adhesive tape.

10. A method of protecting a digit, comprising the steps of:
adhesively mounting a foam member on a protective member having an initial, generally planar, rigid shape;
forming a plurality of grooves in the protective member for enabling the protective member to be flexed and bent to a curved shape;
mounting the protective member and the foam member on an adhesive tape;
flexing and bending the protective member to the curved shape; and
mounting the foam member and the protective member in the curved shape on, and about, the digit to be protected.

11. The method of claim 10, and constituting the protective member of a plastic material with opposite peripheral edges, and forming the grooves in mutual parallelism between the edges.

12. The method of claim 10, and configuring each groove with a generally rectangular shape.

13. The method of claim 10, and configuring the protective member with a generally square shape with rounded corners.

14. The method of claim 10, and configuring the protective member with opposite surfaces, and forming the grooves to extend from one of the surfaces through the protective member, and to terminate short of the other of the surfaces to form an outer skin.

15. The method of claim 10, and ventilating the protective member.

16. The method of claim 10, and configuring the protective member with generally rectangular ribs bounding each groove.

17. The method of claim 10, and mounting peelable strips on the adhesive tape for removal therefrom.

18. The method of claim 10, and wrapping a bandage around the digit underneath the protective member, the foam member and the adhesive tape.
